(19) United States
(12) Patent Application Publication
(10) Pub. No.: US 2024/0008720 A1
(43) Pub. Date: Jan. 11, 2024

(54) ELONGATE ENDOSCOPIC COVERING

(71) Applicant: GYRUS ACMI, INC. D/B/C OLYMPUS SURGICAL TECHNOLOGIES AMERICA, Westborough, MA (US)

(72) Inventors: Kester Julian Batchelor, Mound, MN (US); Erica Matthes, Waltham, MA (US); Richard K. Lessig, Macungie, PA (US); Anthony R. Pirozzi, Raleigh, NC (US)

(21) Appl. No.: 18/346,615

(22) Filed: Jul. 3, 2023

Related U.S. Application Data
(60) Provisional application No. 63/358,751, filed on Jul. 6, 2022.

(51) Int. Cl. A61B 1/00; A61B 1/31

(52) U.S. Cl. CPC A61B 1/00135; A61B 1/00082; A61B 1/31

(57) ABSTRACT

An elongate covering can be used such as to cover an outer surface of an endoscope. For example, the elongate covering can include or use a flexible thin-walled tube. The thin-walled tube can include a distal end and an open proximal end, and an elongate tubular portion extending therebetween. The elongate tubular portion can be sized and shaped such as to surround and engage an elongate portion of the duodenoscope. The covering can also include or use an expandable stabilizer, such as an inflatable balloon, disposed at a part of the flexible thin-walled tube.

### PRIORITY CLAIM

This application claims the benefit of priority to U.S. Provisional Patent Application Ser. No. 63/358,751, filed Jul. 6, 2022, the contents of which are incorporated herein by reference.

### TECHNICAL FIELD

This document describes generally, among other things, endoscopic techniques for cholangioscopy or other procedures, and more particularly to systems and methods including an elongate covering for a duodenoscope for endoscopic access of a duodenum.

### BACKGROUND

Endoscopes can be used in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices or biological matter collection devices for sampling or treating an anatomical region, and providing suction passageways for collecting fluids (e.g., saline or other preparations or bodily fluids), among other procedures. Examples of such an anatomical region can include a gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

In certain endoscopy procedures, the distal portion of the endoscope can be configured for supporting and orienting another therapeutic device. In some systems, two endoscopes can be used. A first, parent endoscope can be used for guiding a second, child endoscope inserted therein and directing the second endoscope, such as using an elevator located at the distal end of the first endoscope. Such systems can be helpful in guiding an endoscope to anatomic locations that are relatively distant from an insertion location.

Peroral cholangioscopy is an endoscopy technique that permits direct endoscopic visualization, diagnosis, and treatment of various disorders of patient biliary and pancreatic ductal system using miniature endoscopes and catheters inserted through the accessory port of a duodenoscope. Peroral cholangioscopy can be performed by using a dedicated cholangioscope that is advanced through the accessory channel of a duodenoscope, as used in Endoscopic Retrograde Cholangio-Pancreatography (ERCP) procedures. ERCP is a technique that combines the use of endoscopy and fluoroscopy to diagnose and treat certain problems of the biliary or pancreatic ductal systems, including the liver, gallbladder, bile ducts, pancreas, or pancreatic duct. In ERCP, a cholangioscope (here acting as an auxiliary scope, or a “daughter” scope) can be attached to and advanced through a working channel of a duodenoscope (here acting as a primary scope, or a “parent” scope). In ERCP, biliary cannulation can be achieved directly with the tip of the cholangioscope alone or passed over a guidewire such as to promote ease and control of certain procedures. A tissue retrieval device can be inserted through a channel of the cholangioscope such as to address biological matter (e.g., gallstones, bill duct stones, cancerous tissue) or to manage a stricture or blockage in bile duct.

### SUMMARY

This document describes generally, among other things, an instrument covering for endoscopic procedures (which can refer generally to include endoscopic, arthroscopic, laparoscopic or similar minimally-invasive procedures). The instrument covering may include a removable covering for an endoscope having an expandable stabilizer for anchoring the endoscope to patient anatomy during a procedure. For example, devices and methods described herein can include device could be used to help stabilize a duodenoscope such as in an ERCP procedure.

The present inventors have recognized challenges with certain medical devices, and in particular endoscopes and duodenoscopes. Such challenges can include, among other things, 1) the difficulty in navigating an endoscope to a difficult-to-reach anatomic location, 2) the increased time and associated cost of navigating an endoscope to an incorrect location, and 3) the possibility of potential tissue effects of impacting sensitive tissue with an endoscope. Such problems can be particularly challenging in duodenoscopy procedures, in which an auxiliary scope (also referred to as daughter scope, or cholangioscope) can be attached and advanced through the working channel of a “main scope” (also referred to as a mother scope or duodenoscope). This document describes an endoscopic technique that can include using a covering, such as including an expandable stabilizer, such as to help orient and hold an endoscope, such as a duodenoscope, within patient anatomy during a procedure. This document also describes a covering that can be formed such as to adhere to an endoscope while avoiding or mitigating excessive attachment to patient anatomy upon device removal. For example, an endoscope such as a duodenoscope, at least partially enveloped by the elongate covering, can be inserted through a patient's mouth, down the esophagus, through the stomach, into the duodenum. The elongate covering can include a port. The port can allow visualization out a distal tip of the duodenoscope. The port can also allow for passage of a secondary device, such as a cholangioscope, through an access lumen of the duodenoscope and out of the port. In an example, this can help allow a cholangioscope to be passed through the ampulla of vater, and into the bile ducts or the pancreatic ducts, if desired.

An elongate covering can be used such as to cover an outer surface of an endoscope. In an example, the endoscope can include a duodenoscope for use during endoscopic access of a duodenum. For example, the elongate covering can include or use a flexible thin-walled tube. The thin-walled tube can include a distal end and an open proximal end, and an elongate tubular portion extending therebetween. The elongate tubular portion can be sized and shaped such as to surround and engage an elongate portion of the duodenoscope. The covering can also include or use an expandable stabilizer, such as can be disposed at a part of the flexible thin-walled tube. For example, the expandable stabilizer can be expandable laterally with respect to the elongate tubular portion. The expandable stabilizer can be expandable to contact or protrude against patient anatomy such as to help stabilize the flexible thin-walled tube with respect to the patient anatomy.

The elongate covering can include or use an end cap. The end cap can extend distally from the flexible thin-walled tube at the distal end. The distal end of the flexible thin-walled tube can be open-ended and attachable to the elongate portion of the duodenoscope. For example, the flexible thin-walled tube can be clipped place to a part of the duodenoscope at one or more locations. Also, the flexible thin-walled tube can be elastically connected at the distal end or along the length of the elongate portion of the duodenoscope. The end cap can be at least partially formed of a transparent material and can include a side port. The side port can be sized and shaped such as to permit lateral exit of an instrument extending through the duodenoscope. In an example, the end cap can include or use at least one coupling feature that can be sized and shaped to interface with a distal end of a duodenoscope. In an example, the expandable stabilizer can include an inflatable balloon such as can be configured to expand against the duodenum. The balloon can vary between an inflated state and a deflated state, such as based on an inflation pressure that can be supplied by an external source. For example, the elongate covering can include or use an elongate fluid-communicating vessel extending between the inflatable balloon and the proximal end of the flexible thin-walled tube. Herein, fluid-communicating refers to fluid including gas or liquid.

The elongate tubular portion can include one or more features that can help inhibit or prevent slipping, bunching, or separation of the elongate covering from the endoscope. For example, an interior surface of the elongate tubular portion can include a plurality of friction features. The friction features can be configured to engage the elongate portion of the duodenoscope. For example, an interior surface of the elongate tubular portion can be formed such that the interior surface has a greater coefficient of static friction than an exterior surface of the elongate tubular portion. The plurality of friction features can include one or more ribbing features along a length of the interior surfaces of the elongate tubular portion.

The elongate covering can include or use a liquid lubricant, a medicament, or a biocide, such as on an exterior surface of the elongate tubular portion. The elongate covering can include or use a water-activated hydrophilic coating, a hydrophobic coating, or an antibiotic coating included on an exterior surface of the elongate tubular portion.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A and FIG. 1B depict an example of an elongate covering to cover an elongate portion of an endoscope.

FIG. 2A and FIG. 2B depict an example of an elongate covering including an expandable stabilizer varying between an expanded position and a retracted position.

FIG. 3A and FIG. 3B depict an example of an elongate covering including an expandable stabilizer varying between an expanded position and a retracted position.

FIG. 4 is a schematic diagram of an example of an endoscopy system.

FIG. 5 depicts a distal portion of an example of an elongate covering enclosing a duodenoscope positioned in a patient duodenum.

FIG. 6 is a flowchart showing a method of using an endoscopic system.

FIG. 7 is a flowchart indicating a reprocessing method for the elongate covering.

### DETAILED DESCRIPTION

This document describes, among other things, an endoscope, such as for one or more endoscopic procedures (which can refer generally to include endoscopic, arthroscopic, laparoscopic or similar minimally-invasive procedures). In an approach to endoscopy at a location inferior to a patient's pylorus, e.g., Endoscopic retrograde cholangiopancreatography (ERCP), an auxiliary scope (also referred to as daughter scope, or cholangioscope) can be attached and advanced through the working channel of a “main scope” (also referred to as mother scope or duodenoscope). Here, the main scope can include optics and can be manipulatable via user controls such as to navigate patient anatomy. For example, the main scope can be inserted at a patient esophagus and operated or manipulated to travel towards a patient duodenum. After a distal end of the main scope is placed at or near a target site within the patient, such as aided by the main scope optics, the auxiliary scope can be fed through a channel of the main scope, such as to treat the target site. ERCP procedure can be combined with a cholangioscopy procedure, such as in which one or more guide wires, cannula or various therapeutic or diagnostic devices can be deployed into the Common Bile Duct (CBD). However, introducing a device into the CBD is technically very challenging. This is because the duodenoscope, itself, supports the cholangioscope. Controlling the two scopes can be demanding of a physician. In such procedures, it is challenging to orient and place the duodenoscope with respect to patient anatomy and to maintain a desired placement throughout the course of the procedure. Further, some approaches to secure the duodenoscope with respect to patient anatomy can introduce challenges in device removal following the procedure.

The present inventors have realized techniques for endoscopic treatment for difficult-to-reach locations. This can include providing an elongate covering of a portion of the scope, such as with an expandable stabilizer. This can help mitigate challenges of placing, securing, and removing an endoscope such as a duodenoscope. An elongate covering such as described herein can also help reduce device maintenance. The elongate covering can provide an opening, such as a side port, out of the duodenoscope, such as for visualization and secondary device passage.

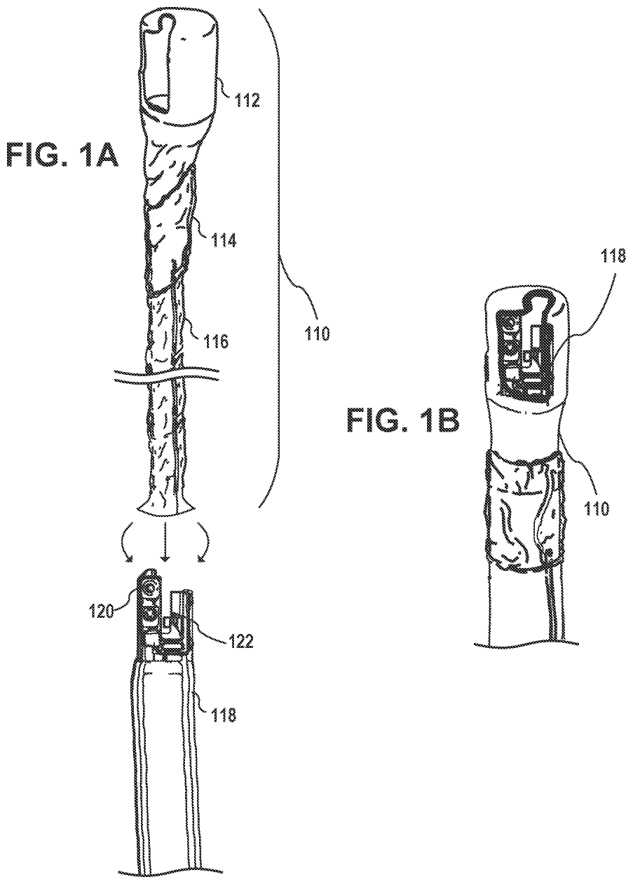

FIG. 1A and FIG. 1B depict an example of an elongate covering to cover an elongate portion of an endoscope. For example, an elongate covering 110 can include or use an end cap 112, an expandable stabilizer 114, and an elongate tubular portion or sleeve 116. In an example, the elongate tubular portion 116 is formed at a length within a range of about 100 cm and about 150 cm for covering a desired length of an elongate portion of an endoscope 118. In an example, the endoscope 118 can be a duodenoscope for use during endoscopic access of a patient duodenum. The endoscope 118 can include or use an endoscope tip at a distal end of the endoscope 118, and the endoscope tip can include an optics unit 120 and a secondary device guide or passage 122.

The end cap 112 can be sized and shaped such as to cover the tip at the distal end of the endoscope 118, such as to cover the optics unit 120 and the secondary device passage 122 without constraining visualization or access of the access lumen of the endoscope 118. As depicted in FIG. 1B, once paired with the distal end of the endoscope, the end cap 112 can at least partially encapsulate the tip at the distal end of the endoscope 118. For example, the end cap 112 can help protect an endoscopic mechanism within the tip at the distal end. The end cap 112 also can help reduce a risk of infection such as by reducing detritus collection on the endoscope during a procedure. Also, an outer surface of the end cap 112 can be formed of a soft material and with an atraumatic edge such as to help promote ease of inserting the endoscope into the patient anatomy and to help promote atraumatic contact of the end cap 112 to the patient anatomy. The end cap 112 can be at least partially formed of a transparent material and can include a side port. Herein, “transparent” materials can allow viewing therethrough to a human user at visible wavelengths of light. In another example, the end cap 112 can be formed of a translucent or opaque material. The end cap 112 can be formed of a transparent or semi-transparent polymer such as polycarbonate, polyethylene terephthalate (PET), polymethyl methacrylate (PMMA), Amorphous Copolyester (PETG), Polyvinyl Chloride (PVC), and the like. The side port can be sized and shaped such as to permit lateral exit of the secondary instrument extending through the endoscope 118. In an example, the end cap 112 can include or use at least one coupling feature that can be sized and shaped to interface with or mechanically engage a distal end of the endoscope. The coupling feature can include threading, a lip, a groove, a follower, a snap-fit feature, a detent, a clip, a magnet, a hook, a tab, a latch, a snap, or other suitable mechanical connectors.

The elongate tubular portion 116 can include one or more features that can help inhibit or prevent slipping, bunching, or separation of the elongate covering from the endoscope 118. For example, an interior surface of the elongate tubular portion 116 include a plurality of friction features such as can be configured to engage the elongate portion of the endoscope 118. The plurality of friction features can include one or more ribs, studs, or other texturing features along a length of the interior surfaces of the elongate tubular portion 116. Also, the plurality of friction features can include a coating such as a thermoplastic elastomer (TPE). In an example, an interior surface of the elongate tubular portion 116 can be formed such that the interior surface has a greater coefficient of static friction than an exterior surface of the elongate tubular portion 116. Also, the interior surface of the elongate tubular portion 116 can be formed, by way of one or a combination of one or more material properties, one or more lubricants, or one or more texturing features such as can be included in the elongate tubular portion, such as to provide a greater coefficient of static friction on an interior surface than on an exterior surface of the elongate tubular portion 116. In an example, the interior surface of the elongate tubular portion 116 can have a coefficient of static friction between about 0.50 and 0.90 at an ambient temperature of 40 degrees Celsius, e.g., greater than 0.70 at an ambient temperature of 40 degrees Celsius. The elongate tubular portion 116 can include or use a liquid lubricant, a medicament, or a biocide, on an exterior surface of the elongate tubular portion 116. Additionally or alternatively, the elongate tubular portion 116 can include or use a water-activated, hydrophilic coating, a hydrophobic coating, or an antibiotic coating included on an exterior surface of the elongate tubular portion 116. The elongate covering can be formed of a disposable material formed for a single-use application, if desired. Also, the elongate covering can be reusable and able to be sanitized for several subsequent procedures, if desired.

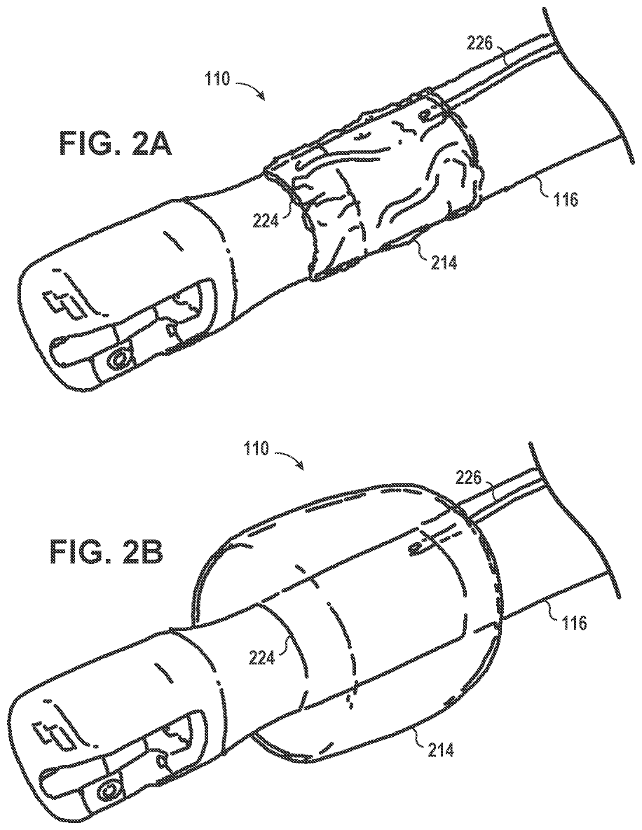

As explained further below with respect to FIG. 2A and FIG. 2B, the expandable stabilizer can vary between a laterally expanded position and a laterally retracted position. In an example, such as when the expandable stabilizer is in the retracted position, the elongate covering 110 can include or use an elastic section. The elastic section, which can be formed in the elongate tubular portion 116 or the expandable stabilizer 114, can form a smaller inner diameter than an outer diameter of the elongate portion of the endoscope 118. The elongate tubular portion 116 can be a flexible, thin-walled tube. For example, the smaller inner diameter of the elongate tubular portion 116 can be stretchable around the larger diameter of the elongate portion of the endoscope 118. The elongate tubular portion 116 can have a wall thickness that is between about 0.01 mm and about 0.2 mm. For example, elongate tubular portion 116 can have a wall thickness that is between about 0.1 mm and about 0.175 mm, such as a wall thickness less than about 0.15 mm.

FIG. 2A and FIG. 2B depict an example of an elongate covering including an expandable stabilizer varying between an expanded position and a retracted position. The expandable stabilizer, such as a stabilizing balloon 214, can be disposed within or otherwise integrated within the elongate tubular portion 116. Alternatively or additionally, the expandable stabilizer can be attached to the elongate tubular portion 116 such as using one or more of radiofrequency welding, solvent, glue, or other medical adhesive. The expandable stabilizer can be expandable laterally with respect to the elongate tubular portion 116. The elongate stabilizer can be expandable such as to contact or protrude against patient anatomy, such as a patient duodenum. In turn, the elongate tubular portion 116 and the expandable stabilizer can work together to grasp the endoscope 118 (as depicted in FIG. 1A) and secure it with respect to patient anatomy such as to maintain device placement during a procedure.

As depicted in FIG. 2A and FIG. 2A, the expandable stabilizer can include or use the stabilizing balloon 214. The stabilizing balloon 214 can be inflated to a diameter large enough to create sufficient to contact the inner walls between the stabilizing balloon 214 and a desired portion of the patient anatomy. In an example, the stabilization balloon 214 can be generally toroid shaped. The elongate tubular portion 116 can extend through an opening of the toroid-shaped stabilizing balloon 214. For example, the opening 224 of the toroid can be offset from a longitudinal axis of the elongate tubular portion 116, such as to be asymmetric. Alternatively, the opening 224 of the toroid can be approximately centered with respect to the longitudinal axis of the elongate tubular portion 116. In another example, the stabilizing balloon 214 can include a fluid enclosing pocket of material attached to an outer surface or an inner surface of the elongate tubular portion 116. Here, the stabilizing balloon 214 can include a section of an inner surface of the elongate tubular portion 116 or a section of an outer surface of the elongate tubular portion 116. The stabilizing balloon 214 can be rated such as to be maximally inflatable to a pressure between about 1 cmH₂O and 30 cmH₂O. For example, the stabilizing balloon 214 can be rated such as to be maximally inflatable to a pressure between about 8 cmH₂O and about 12 cmH₂O. Also, the stabilization balloon 214, in an inflated state, can have an outer diameter between about 20 cm and 45 cm. For example, stabilization balloon 214, in an inflated state, can have an outer diameter between about 26 cm and about 38 cm. In an example, the stabilization balloon 214 can be fluidly connected to an inflation source via an elongate fluid-communicating vessel, such as an inflation line 226, and the inflation line 226 can be included in or used by the elongate covering 110. For example, the inflation line 226 can be at least partially integrated inside the elongate covering 110. In an example, the inflation line 226 can include or use or be fluidly connected to an endotracheal (ET) tube. Also, the elongate covering 110 can include a plurality of stabilizing balloons 214. For example, a plurality of stabilizing balloons 214 can be included on different sections of the elongate tubular portion 116. In operation and use, the plurality of stabilizing balloons can be at multiple locations along the anatomical route, such as at or near the stomach, the duodenal cap, the duodenal circular rings, or esophagus. Alternatively or additionally, two or more stabilizing balloons 214 can be included adjacent to each other at approximately the same section of the elongate tubular portion 116. Where the elongate covering 110 includes a plurality of stabilizing balloons 214, each of the stabilizing balloons 214 can be independently inflatable or deflatable, such as via a plurality or respective separate inflation lines 226, to manipulate the elongate covering 110 and the endoscope within the patient anatomy.

The expandable stabilizer can be actuated to alter between a laterally expanded, stabilizing position, wherein the elongate covering 110 is anchored to the surrounding patient anatomy, and a laterally retracted, released position where the elongate covering 110 (and the endoscope contained therein) is manipulatable within the patient anatomical route. For example, when the distal portion of the endoscope is placed in a desired position in the anatomical route, the stabilization balloon 214 can be inflated to contact inner walls of the patient anatomy, such as a patient duodenum. In an example where the endoscope is a duodenoscope, the inflated stabilization balloon 214 can help inhibit or prevent the distal portion of the duodenoscope from advancing further than desired into the duodenum or to help inhibit or prevent the duodenoscope from unintentionally withdrawing back into the stomach during the procedure.

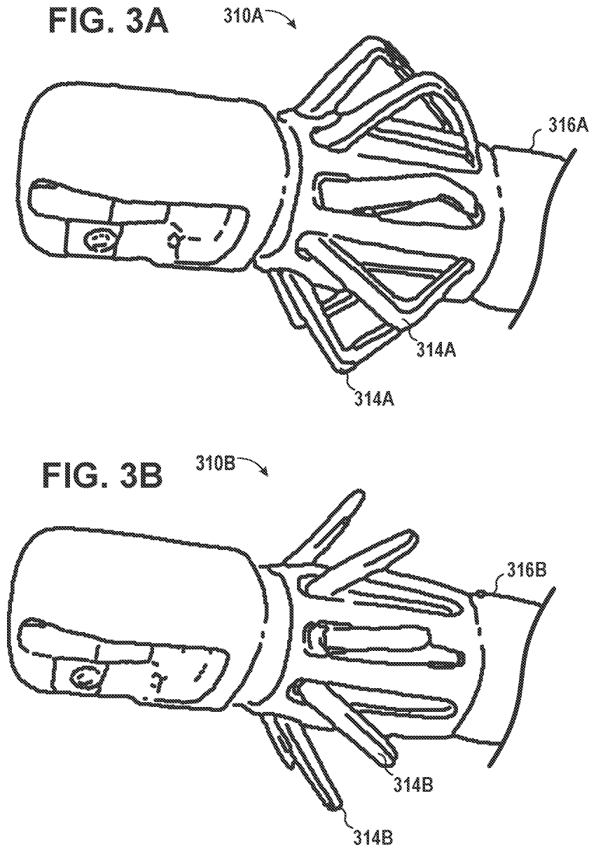

FIG. 3A and FIG. 3B depict an example of an elongate covering including an expandable stabilizer varying between an expanded position and a retracted position. Here, the expandable stabilizer can include a plurality of expandable wings or folds 314A, or expandable arms 314B such as for contacting patient anatomy such as to help stabilize elongate coverings 310A and 310B and the endoscope within the anatomical route. The folds 314A and arms 314B can be attached to the respective elongate tubular portions 316A and 316B in a similar fashion to that previously described with respect to the stabilizing balloon 214 (as depicted in FIG. 2A and FIG. 2B). The folds 314A and arms 314B can be made of a soft, atraumatic material radial arms and can be activated via a cable mechanism. The folds 314A and arms 314B can be formed of a synthetic material such as a plastic or other polymer. Alternatively, the folds 314A and arms 314B can be formed of a superelastic or other shape-memory material, such as nitinol. The folds 314A and arms 314B can be deployed once in a particular place within the anatomical route. Also, the folds 314A and arms 314B can be constantly deployed, but malleable.

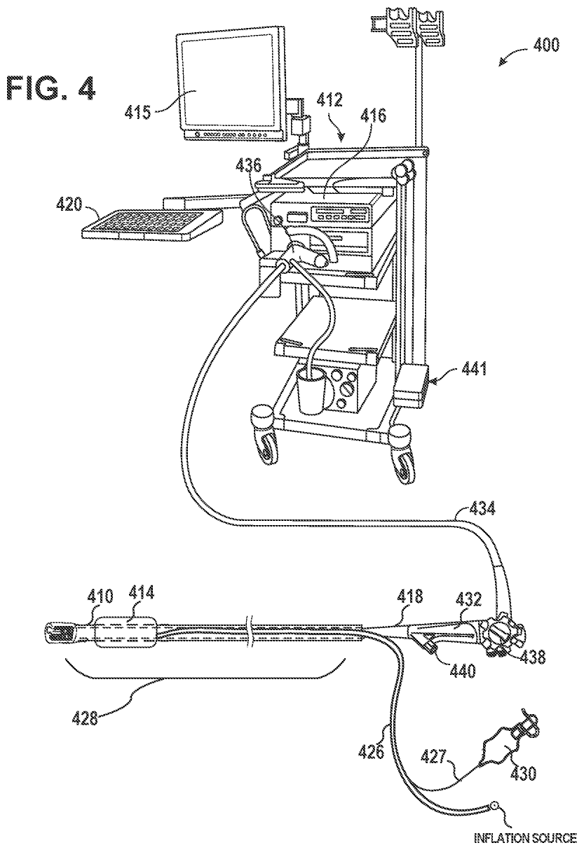

FIG. 4 is a schematic diagram of an example of an endoscopy system. An endoscopy system 400 can include or use an imaging and control system 412, a duodenoscope 418, and an elongate covering 410. The system of FIG. 4 is an illustrative example of an endoscopy system suitable for use with the systems, devices, and methods described herein, such as diagnostic devices or therapy devices. For example, the duodenoscope 418 can be insertable into an anatomical region for imaging and/or to provide passage of one or more sampling devices such as for biopsies, or one or more therapeutic devices for treatment of a disease state associated with the anatomical region. The duodenoscope 418 can interface with and connect to an imaging and control system 412.

The imaging and control system 412 can include or use a controller 416, an output unit 415, and an input unit 420. Imaging and control system 412 can include various ports such as for coupling with the endoscopy system 400. For example, the controller 416 can include a data input and output port for receiving data from and communicating data to the duodenoscope 418. An output unit 415 and an input unit 420 can be used by an operator of the endoscopy system 400 to control one or more functions of the endoscopy system 400 and to view output of the duodenoscope 418. The controller 416 can additionally be used to generate one or more signals or other outputs from treating the anatomical region into which the duodenoscope 418 is inserted. The controller 416 can generate electrical output, acoustic output, a fluid output, or the like such as for treating the anatomical region with, for example, cauterizing, cutting, freezing, or the like.

The duodenoscope 418 can be covered with the elongate covering 410 such as to define an insertion portion 428. The insertion portion 428 can include a distal portion of the duodenoscope which can extend distally from a handle portion 432. Also, the handle portion 432 can be coupled to a cable section 434 and coupler section 436.

The cable section 434 can extend proximally from handle portion 432. The insertion portion 428 can also include one or more working channels (e.g., an internal lumen) that can be elongate and can support inserting one or more therapeutic tools of the distal portion, such as a cholangioscope. The working channel can extend between handle portion 432 and distal portion. Additional utilities, such as fluid passages, guide wires, and pull wires can also be provided by the insertion portion 428 (e.g., via suction or irrigation passageways, or the like).

The handle portion 432 can include the one or more controls 438 and one or more ports 440. The one or more controls 438 can include a knob, dial, switch, or other suitable controls for ergonomic actuation by a user. The one or more controls 438 can also be coupled to a pull wire extending through the insertion portion 428. The one or more ports 440 can couple with, e.g., electrical cables, fluid tubes and the like to handle portion 432 for coupling with the insertion portion 428.

The endoscopy system 400 can include a stabilization balloon 414 of the elongate covering 410, an inflation line 426, such as the ET tube, and an inflation monitor 430. The inflation monitor 430 can be a pilot balloon tethered to the inflation line 426 via a pilot balloon line 427 such as to indicate whether the stabilization balloon 424 is in an inflated state or a deflated state. In an example, the inflation monitor 430 can indicate a level of inflation of the stabilization balloon. For example, where the inflation monitor 430 is a pilot balloon, the pilot balloon can indicate the level of inflation depending on a rigidity of the pilot balloon something like this pilot balloon.

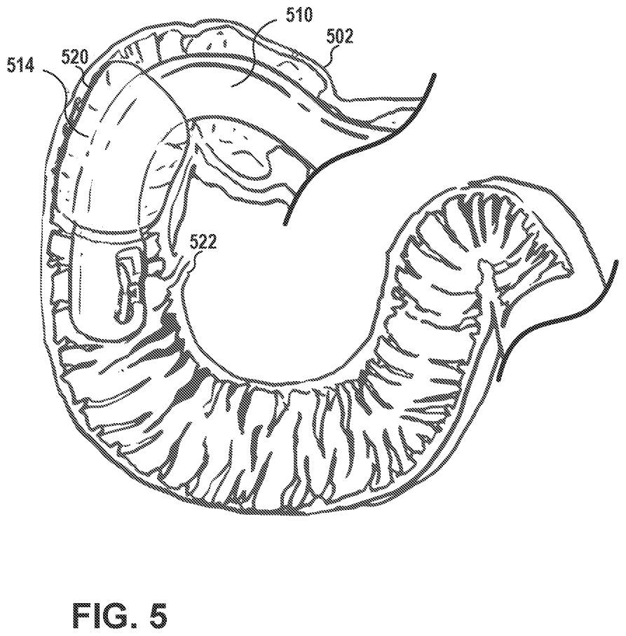

FIG. 5 depicts a distal portion of an example of an elongate covering 510 enclosing a duodenoscope positioned in a patient duodenum 502. A patient duodenum 502 can include duct wall 520, sphincter of Oddi 522. The duodenum 502 includes an upper part of the small intestine. As previously described, the elongate covering 510 can include or use an expandable stabilizer such as a stabilization balloon 514, and the stabilization balloon 514 can be inflated such as to contact the duct wall 520 to anchor the elongate covering 510 enclosing the duodenoscope.

In certain endoscopic procedures, the elongate covering 510 enclosing the duodenoscope can provide a passage such as to guide a cholangioscope towards or through a patient duodenum 502. For example, the cholangioscope can be guided through the duodenoscope and into or past the sphincter of Oddi 522. Therefrom, a physician operating the cholangioscope can navigate the cholangioscope toward a patient gall bladder or liver to perform various procedures.

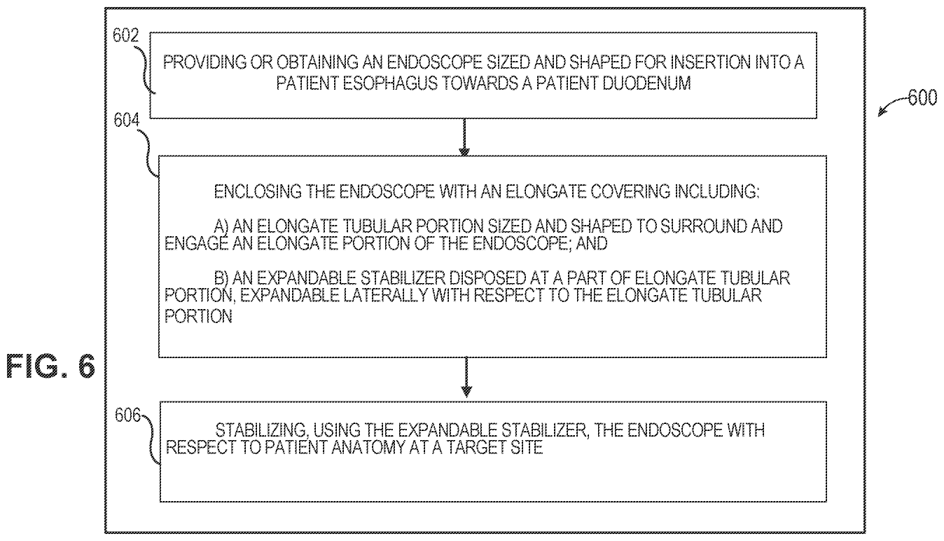

FIG. 6 is a flowchart showing a method of using an endoscopic system. At 602, an endoscope such as a duodenoscope can be provided or obtained. The endoscope can be sized and shaped such as for insertion into a patient esophagus towards a patient duodenum. At 604, the endoscope can be enclosed with an elongate covering. The elongate covering can include a flexible thin-walled tube including a distal end and an open proximal end, and an elongate tubular portion extending therebetween. The elongate tubular portion can be sized and shaped such as to surround and engage an elongate portion of the endoscope. The elongate covering can also include or use an expandable stabilizer. The expandable stabilizer can be disposed at a part of the flexible thin-walled tube, expandable laterally with respect to the elongate tubular portion.

At 606, the endoscope can be stabilized with respect to patient anatomy at a target site using the expandable stabilizer. For example, an inflatable balloon of the elongate portion can be inflated such as to contact the patient anatomy, e.g., a patient duodenum. The inflatable balloon can be deflated such as to retract contact with the patient anatomy and release the endoscope from the anatomy such as for removal from the anatomical route. In an example, a daughter scope can be received through an access lumen of the duodenoscope. Also, the daughter scope can be passed through an opening, such as a side port, included in an end cap of the elongate covering.

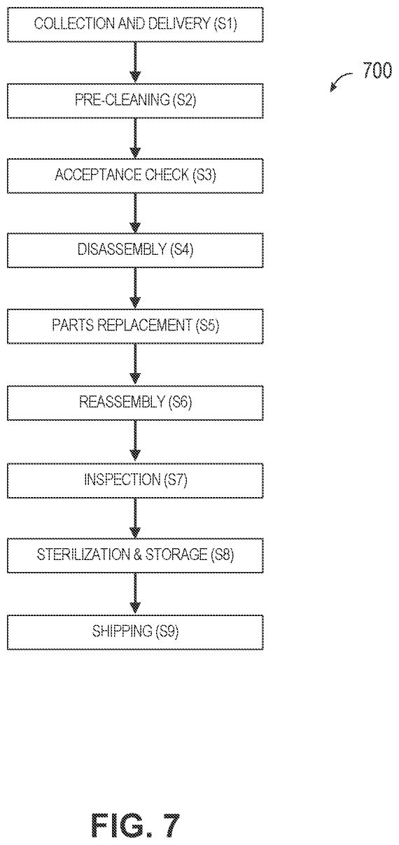

FIG. 7 is a flowchart indicating a reprocessing method for the elongate covering. An elongate covering described above may be disposed of after one use, or may be repeatedly used a plurality of times. In the case of a configuration that is repeatedly used a plurality of times, for example, reprocessing method 700 shown in FIG. 7 is required. An operator collects the used elongate covering after it has been used for treatment and transports it to a factory or the like (Step S1). Then, the operator cleans and sterilizes the collected and transported used elongate covering (Step S2). Next, the operator performs an acceptance check of the used elongate covering (Step S3). Subsequently, the operator disassembles the used elongate covering (Step S4) and replaces some parts of the used elongate covering with new parts (Step S5). After step S5, the operator assembles a new elongate covering (Step S6). In some examples, Step S6 can include adding an identifier to indicate the device has been modified from its original condition, such as a adding a label or other marking to designate the device as reprocessed, refurbished or remanufactured. After Step S6, the operator sequentially performs an inspection (Step S7), sterilization and storage (Step S8), and shipping (Step S9) of the new elongate covering. For example, an elongate covering can include few moving parts, electronics, or mechanisms. Therefore, there is an advantage that the elongate covering may need less attention to, or permit omission of parts replacement (Step S5) and reassembly (S6).

The following, non-limiting examples, detail certain aspects of the present subject matter to solve the challenges and provide the benefits discussed herein, among others.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A disposable elongate covering for a duodenoscope for use during endoscopic access of a duodenum, the covering comprising:
a flexible thin-walled tube including a distal end and an open proximal end, and an elongate tubular portion extending therebetween and sized and shaped to surround and engage an elongate portion of the duodenoscope;
an expandable stabilizer disposed at a part of the flexible thin-walled tube, expandable laterally with respect to the elongate tubular portion, the stabilizer configured to be expanded to stabilize the flexible thin-walled tube with respect to the duodenum; and
an end cap extending distally from the flexible thin-walled tube at the distal end, the end cap including a port sized and shaped to permit lateral exit of a distal portion of a daughter scope extending through the duodenoscope.

2. The disposable elongate covering of claim 1, wherein the end cap includes at least one coupling feature sized and shaped to interface with a distal end of a duodenoscope.

3. The disposable elongate covering of claim 1, wherein the end cap includes a transparent material configured to allow visualization therethrough.

4. The disposable elongate covering of claim 1, wherein the expandable stabilizer includes an inflatable balloon configured to expand against the duodenum, the balloon configured to vary between an inflated state and a deflated state.

5. The disposable elongate covering of claim 4, further comprising an elongate fluid-communicating vessel extending between the inflatable balloon and the proximal end of the flexible thin-walled tube.

6. The disposable elongate covering of claim 4, wherein the inflatable balloon is generally toroid-shaped, the flexible thin-walled tube extending into an opening of the toroid-shaped balloon.

7. The disposable elongate covering of claim 6, wherein the flexible thin-walled tube extends into toroidal opening of the balloon offset from a toroidal central axis of the balloon.

8. The disposable elongate covering of claim 4, wherein the balloon rated to be maximally inflatable to a pressure between 1 cmH₂O and 30 cmH₂0

9. The disposable elongate covering of claim 4, wherein the balloon, in the inflated state, has an outer diameter between 20 cm and 45 cm.

10. The disposable elongate covering of claim 1, wherein an interior surface of the elongate tubular portion having a coefficient of static friction that is greater than 0.70 at an ambient temperature of 40 degrees Celsius.

11. The disposable elongate covering of claim 1, wherein an interior surface of the elongate tubular portion include a plurality of friction features configured to engage the elongate portion of the duodenoscope.

12. The disposable elongate covering of claim 11, wherein the plurality of friction features includes a plurality of ribbing features along a length of the interior surfaces of the elongate tubular portion.

13. The disposable elongate covering of claim 1, further comprising at least one of a liquid lubricant, a medicament, or a biocide, included on an exterior surface of the elongate tubular portion or the expandable stabilizer.

14. The disposable elongate covering of claim 1, further comprising one of: a water-activated hydrophilic coating, a hydrophobic coating, or an antibiotic coating included on an exterior surface of the elongate tubular portion or the expandable stabilizer.

15. The disposable elongate covering of claim 1, wherein an interior surface of the elongate tubular portion has a greater coefficient of static friction than an exterior surface of the elongate tubular portion.

16. An endoscopy system for accessing anatomy inferior to a pylorus of a stomach of a patient, the system comprising:
an endoscope sized and shaped for insertion into a patient esophagus towards a patient duodenum, the endoscope including a proximal portion and a distal portion, and an access lumen extending therebetween, the access lumen providing an inner diameter to receive and pass a secondary medical instrument therethrough; and
an elongate covering for the endoscope, the covering including:
a flexible thin-walled tube including a distal end and an open proximal end, and an elongate tubular portion extending therebetween and sized and shaped to surround and engage an elongate portion of the endoscope;
an expandable stabilizer disposed at a part of the flexible thin-walled tube, expandable laterally with respect to the elongate tubular portion, the stabilizer configured to be expanded to stabilize the flexible thin-walled tube with respect patient anatomy at a target site.

17. The endoscopy system of claim 16, wherein the elongate covering includes an end cap extending distally from the flexible thin-walled tube at the distal end, the end cap and including a side port sized and shaped to permit view or entry out of the endoscope and into the target site of the patient anatomy.

18. The endoscopy system of claim 16, wherein the expandable stabilizer includes an inflatable balloon configured to expand against the duodenum, the balloon configured to vary between an inflated state and a deflated state.

19. The endoscopy system of claim 18, wherein the elongate tubular portion, when the balloon is in the deflated state, includes an elastic section wherein:
the inner diameter of the elongate tubular portion is less than an outer diameter of the elongate portion of the endoscope; and
the smaller inner diameter of the elongate tubular portion is stretchable around the larger diameter of the elongate portion of the endoscope.

20. An endoscopy method for accessing anatomy inferior to a pylorus of a stomach of a patient, the method comprising:
providing or obtaining an endoscope sized and shaped for insertion into a patient esophagus towards a patient duodenum;
enclosing the endoscope with an elongate covering, enclosing including:
a flexible thin-walled tube including a distal end and an open proximal end, and an elongate tubular portion extending therebetween and sized and shaped to surround and engage an elongate portion of the endoscope; and
an expandable stabilizer disposed at a part of the flexible thin-walled tube, expandable laterally with respect to the elongate tubular portion, the stabilizer configured to be expanded to stabilize the flexible thin-walled tube with respect patient anatomy at a target site; and
stabilizing, using the expandable stabilizer, the endoscope with respect to patient anatomy.
